(19) United States
(12) Patent Application Publication
(10) Pub. No.: US 2019/0269810 A1
(43) Pub. Date: Sep. 5, 2019

(54) MEDICAL DEVICE

(71) Applicant: FRESENIUS MEDICAL CARE DEUTSCHLAND GMBH, Bad Homburg (DE)

(72) Inventor: Winfried BREHM, Hofheim (DE)

(73) Assignee: FRESENIUS MEDICAL CARE DEUTSCHLAND GMBH, Bad Homburg (DE)

(21) Appl. No.: 16/347,019

(22) PCT Filed: Oct. 27, 2017
(86) PCT No.: PCT/EP2017/001265

§ 371 Date: May 2, 2019

(30) Foreign Application Priority Data
Nov. 2, 2016 (DE) 10 2016 013 087.3

(51) Int. Cl. A61L 2/10; A61M 1/14; A61L 2/232

(52) U.S. Cl. CPC A61L 2/10; A61M 1/14; A61L 2202/14; A61M 2209/10; A61L 2202/24; A61L 2/232

(57) ABSTRACT

The present invention relates to a medical device, in particular a blood treatment device and in particular a dialysis machine, having at least one handle arrangement, in particular a handle arrangement for a door, wherein the handle arrangement has at least one handle that is configured in ring shape; and wherein the handle arrangement furthermore has at least one disinfection unit that works with light, preferably with UV light, and that is arranged to disinfect the handle, with the handle arrangement furthermore having at least one drive unit that is configured to move the handle along the disinfection unit.

The present invention relates to a medical device having a handle arrangement, in particular having a handle arrangement for a door of the medical device, wherein the medical device preferably is a blood treatment device and particularly preferably a dialysis machine.

The ensuring of sterility is in particular of great importance in the medical sector. It is thus known that a plurality of germs can in particular collect at door handles with which a user actuating the door handle comes into contact, which may possibly have disadvantageous health consequences. Therefore, there is a necessity of disinfection, for which purpose, for example, disinfectant solutions are used that are e.g. applied as a spray.

It is furthermore known from the prior art to carry out a surface disinfection, e.g. by UV light. It is thus known that, for example, UVC or UVA light can develop disinfecting solutions and are therefore suitable for disinfection.

Independently of the fact that possibilities and means for disinfection are already sufficiently known, it is, however, hardly possible in everyday life to disinfect a door handle, for example, after every contact so that there is always a certain risk of infection or contamination on a repeat contact.

It is therefore the underlying object of the present invention to further develop a medical device of the initially named kind such that it is ensured that the surface of the handle is disinfected.

This object is achieved by a medical device having the features of claim 1. Provision is accordingly made that the handle arrangement has at least one handle that is configured in ring shape and that the handle arrangement furthermore has at least one disinfection unit working with light, preferably with UV light, that is arranged to disinfect the handle, wherein the handle arrangement furthermore has at least one drive unit that is configured to move the handle along the disinfection unit such that the handle is acted on by light from the disinfection unit.

It is thus the underlying idea of the present invention to move the handle of the handle arrangement, i.e. the part contacted by the hand to open a door, a flap, a shutter, a window, etc. of the medical device, along one or more disinfection units, for which purpose at least one drive unit is provided that carries out this rotational movement of the handle.

The ring-shaped handle is thus moved along the disinfection unit so that it is ensured that its surface is disinfected.

The movement can be initiated manually by a user, for which purpose at least one actuation element is provided, such as a switch or a contact sensor, to which the handle arrangement is connected or that forms an element of the handle arrangement, with the actuation element being configured such that it activates the drive unit after the actuation by a user. The user thus actuates the actuation element, which has the consequence that the ring-shaped handle is moved along the disinfection unit and in so doing is disinfected at its surface.

Provision is made in a particularly preferred embodiment that at least one sensor, in particular a contact sensor, is provided to which the handle arrangement is connected or that forms an element of the handle arrangement, wherein the sensor is configured to detect the contact or the movement of the handle and/or of an element to be moved by the handle arrangement and to activate the drive unit after such a detection.

In this case, at least one control unit or the like is provided that is connected to the sensor such that said control unit detects the sensor signal and thereupon sets the drive unit into action (simultaneously or with a time offset). This embodiment has the advantage that the user does not have to remember the activation of the disinfection procedure, but that this rather runs in an automated manner.

The sensor or sensors can be arranged at or in the handle arrangement or also at the element such as at a door or window at which the handle arrangement is located. It is thus, for example, also possible to detect that a door has opened, such as by means of a proximity sensor, and then to activate the drive unit.

The sensor is preferably arranged such that it detects a contact or movement of the handle and then sets the drive unit into action.

In accordance with a preferred embodiment of the invention, the disinfection unit is only in operation when the drive unit is also in operation.

In accordance with a further embodiment of the invention, the drive means are only activated with a time offset from the recognition of the handle use. The user can thus actuate the handle e.g. to open the door and the drive unit can subsequently be started.

In a preferred embodiment, the sensor is electrically conductive and is in particular connected to the handle via at least one sliding contact. The handle preferably comprises an electrically conductive material overall or at least in part.

To make a full disinfection possible, the drive unit can be configured such that it moves the handle for so long that each peripheral section of the handle is moved along the disinfection unit at least once i.e. the ring is rotated at least once. It is also conceivable to rotate the handle only so far that the region disposed outside the housing of the handle arrangement before the start of the rotation is disinfected.

To not only disinfect the handle at its outer side or inner side, but rather over the total periphery in the cross-sectional direction, provision can be made that the disinfection unit engages partly or completely around the handle and is preferably itself configured in ring shape. It is also conceivable to provide a plurality of disinfection units in the cross-sectional direction so that this objective is reached.

The at least one disinfection unit is preferably configured such that the handle is completely disinfected on its surface after the disinfection.

To achieve an orderly movement of the handle, provision can be made that one or more guide elements, in particular guide rollers, are present that guide the handle on its movement.

In this respect, at least one of the guide elements can be driven and can thus simultaneously form the drive unit or a part of the drive unit.

It is conceivable that the drive unit is configured such that the handle is moved in a direction in which it is first moved along the guide element or elements and subsequently along the disinfection unit.

The disinfection unit is preferably configured in a manner to emit UVC or UVA light. However, other wavelength ranges are also covered by the invention such as the lower wavelength range of visible light, e.g. a wavelength range between 350 nm and 450 nm.

The at least one disinfection unit can have one or more UV LEDs.

In accordance with a further embodiment of the invention, the handle is hollowed at the inside, which brings along the advantage that the handle is comparatively light and can therefore be moved with a small exertion of force.

Provision can furthermore be made that the handle comprises plastic or metal, preferably stainless steel, overall or at least at its surface.

It is furthermore conceivable that the handle has a surface or a surface coating that contains catalytically or photocatalytically active substances, in particular titanium dioxide. In this case, a further improvement of the disinfecting efficiency is achieved. In this respect, other light sources can also or alternatively be used as UV light sources to develop a disinfecting effect, e.g. light sources that emit light with a wavelength of 380 nm.

The present invention furthermore relates to the use of a handle arrangement in accordance with the characterizing portion of one of the claims 1 to 13 as a door handle for a medical device, in particular for a blood treatment device such as a dialysis machine.

Further details and advantages of the invention will be explained in more detail with reference to an embodiment shown in the drawing.

There are shown:

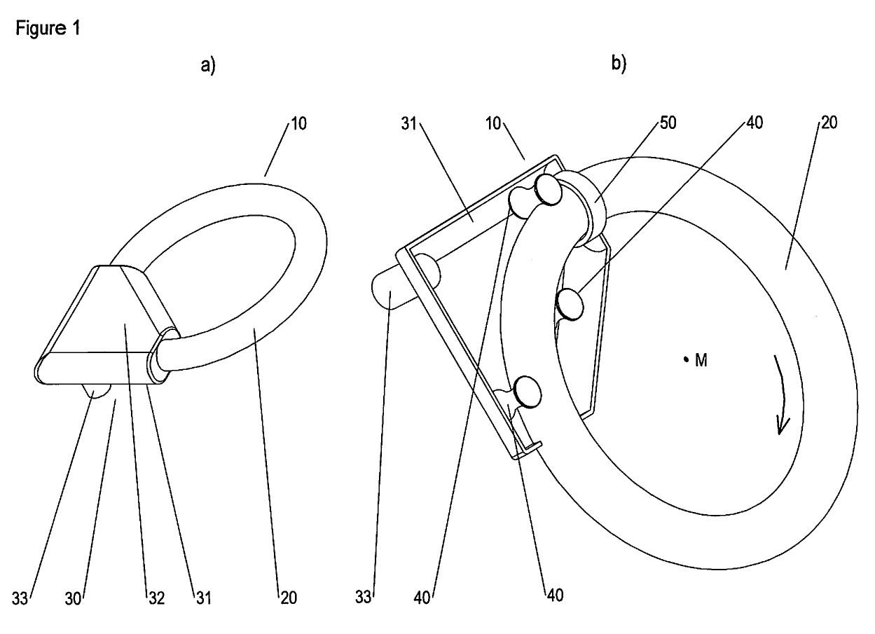

FIG. 1: a perspective view of the handle arrangement with a closed housing and with an open housing;

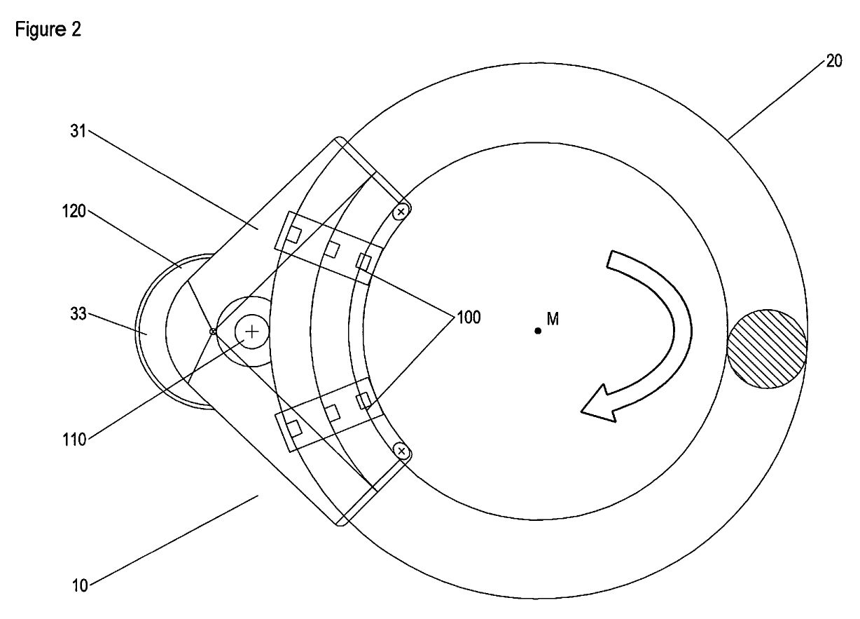

FIG. 2: a schematic view of the handle arrangement in a plan view in a further embodiment; and

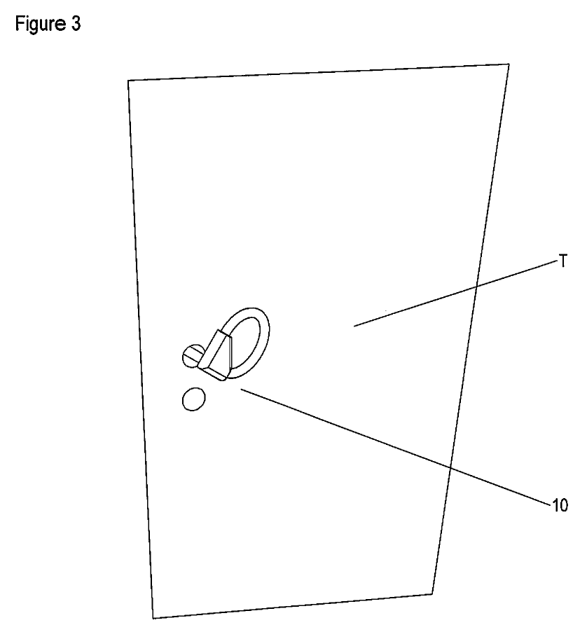

FIG. 3: a perspective view of a door with a handle arrangement in accordance with the invention.

FIG. 1 shows the handle arrangement as a whole with the reference numeral 10.

The handle arrangement comprises a ring-shaped handle 20 that forms the actual handle part to be gripped by the user and that can, for example, be configured as hollow at the inside and that comprises e.g. stainless steel or also another electrically conductive material or another material.

The handle arrangement furthermore comprises a housing 30 that comprises two half-shells that can be latched to one another.

FIG. 1a shows the upper half shell 32 and the lower half shell 31, i.e. the closed housing; and FIG. 1b only shows the lower half shell 31. A fastening element 33 for fixing the handle arrangement 10 to a door or the like is located at the lower half shell.

The housing has an inlet opening and an outlet opening whose size approximately corresponds to that of the outer diameter of the handle and through which the moving handle enters into the housing and exits it.

As can be seen from FIG. 1b, guide rollers 40 are located in the housing 30 that guide the ring-shaped handle 20 along a path that corresponds to the design of the ring.

In the embodiment shown here, three guide rollers 40 are provided, but more or fewer than three guide rollers can also be present.

In general, other guide elements such as rings, webs or the like that guide the handle on its path are also conceivable.

One, more or all the guide rollers 40 are connected to an electric motor (not shown) or have an electric motor that is suitable to set the guide rollers in a rotary movement about their longitudinal axis. By the contact of the guide rollers 40 with the handle 20, the latter is set into a rotary movement in the direction of the arrow, i.e. the handle rotates about a point or center point M, with it being guided by the guide rollers 40.

With a handle that is circular in a plan view, the center point M, i.e. the axis of rotation, is exactly at the circle center point. Other shapes than circular handles such as elliptical designs are also covered by the invention.

The axis of rotation about which the handle is rotated preferably stands perpendicular or at angle to the perpendicular on the plane formed by the handle.

Reference numeral 50 designates a UV light source that is likewise of ring shape ad that completely engages around the handle 20 in its cross-sectional direction. The UV light source 50 is configured such that the ring is acted on by UV light (e.g. UVC light) over the full periphery in the cross-sectional direction.

As can be seen from FIG. 1b, the handle 20 first runs through the region of the guide rollers 40 and then moves through the UV light source 50. The handle section thus disinfected then again leaves the housing 30.

A sensor, not shown, is present in the housing 30 and is electrically conductively connected, e.g. via a sliding contact or a roller, to the handle 20. A control unit, likewise not shown, is provided that is connected to the sensor and to the drive unit and that activates a motor in the housing that rotates the ring or the handle 20 after every contact or actuation of the handle 20.

The control unit can be arranged within or outside the housing.

A capacitive contact sensor can be considered as the sensor, for example.

A sensor within the framework of the present invention is generally any desired element by means of which a contact or an actuation of the handle or of the part such as the door can be detected at which the handle arrangement is located.

After the activation by the control unit, the ring or the handle 20 is rotated at least or exactly so far until the previously exposed region (i.e. not located in the housing) is disinfected. It is thus ensured that the last contacted region is disinfected. On the rotation of the handle 20, its surface is irradiated by means of one or more UV LEDs and is disinfected in so doing.

The irradiation is preferably arranged such that it takes place after the passing of the guide elements, as can be seen from FIG. 1b; a recontamination is thus avoided.

The handle does not have any corners or edges and a complete disinfection of its surface is ensured by its shape. In addition, the ring-shaped handle offers a pleasing and comfortable actuation.

An opening of the door is possible with little contact, for example only by a finger and a slipping from the handle is practically precluded. The ring shape moreover offers the advantage that a simply configured holder corresponding to the size of the ring can be used for its fastening to the door or the like. The irradiation unit can be integrated into the holder in a simple manner.

FIG. 2 shows a further embodiment in which two mutually spaced apart ring-shaped UV LEDs 100 are provided. The roller 110 that is driven by a motor is located between them. The power supply 120 of said motor can generally take place via the axle or via the fastening element by means of which the handle arrangement is fastened or also autonomously, e.g. by means of a battery or by means of a rechargeable battery.

The ring can be circular in its plan view or can also have a shape differing therefrom. This applies accordingly to the cross-section of the ring.

FIG. 3 shows an embodiment, not covered by the present invention, of the handle arrangement at a room door T.

The invention comprises the fixing of the handle arrangement to any desired element of blood treatment devices such as dialysis machines, such as to their housings or to their doors.

1. A medical device, in particular a blood treatment device and in particular a dialysis machine, having at least one handle arrangement, in particular a handle arrangement for a door, characterized in that the handle arrangement has at least one handle that is configured in ring shape; and in that the handle arrangement furthermore has at least one disinfection unit that works with light, preferably with UV light, and that is arranged to disinfect the handle, with the handle arrangement furthermore having at least one drive unit that is configured to move the handle along the disinfection unit.

2. A medical device in accordance with claim 1, characterized in that at least one actuation element is provided to which the handle arrangement is connected or that forms an element of the handle arrangement, with the actuation element being configured such that it activates the drive unit after the actuation by a user.

3. A medical device in accordance with claim 1, characterized in that at least one sensor, in particular a contact sensor, is provided to which the handle arrangement is connected or that forms an element of the handle arrangement, wherein the sensor is configured to detect the contact or the movement of the handle and/or of an element to be moved by the handle arrangement and, with the sensor being configured to activate the drive unit on or after such a detection.

4. A medical device in accordance with claim 3, characterized in that the sensor is electrically conductively connected to the handle, preferably via a sliding contact.

5. A medical device in accordance with claim 2, characterized in that the drive unit is configured to move the handle for so long that each peripheral section of the handle or the previously exposed section of the handle is moved along the disinfection unit at least once.

6. A medical device in accordance with claim 1, characterized in that the disinfection unit engages partly or completely around the handle and is preferably itself configured in ring shape.

7. A medical device in accordance with claim 1, characterized in that one or more guide elements, in particular guide rollers, are provided that guide the handle on its movement.

8. A medical device in accordance claim 7, characterized in that at least one of the guide elements is driven and thus forms the drive unit or a part of the drive unit.

9. A medical device in accordance with claim 7, characterized in that the drive unit is configured such that the handle is moved in a direction in which the handle is first moved along the guide element or elements and subsequently along the disinfection unit.

10. A medical device in accordance with claim 1, characterized in that the disinfection unit is configured to emit UVC light or UVA light.

11. A medical device in accordance with claim 1, characterized in that the handle is hollow at the inside.

12. A medical device in accordance with claim 1, characterized in that the handle comprises plastic or metal, preferably stainless steel, overall or at least at its surface.

13. A medical in accordance with claim 1, characterized in that the handle has a surface or a surface coating that has catalytically or photocatalytically acting substances, in particular titanium dioxide.

14. Use of a handle arrangement in accordance with the characterizing portion of claim 1 as a handle and in particular as a door handle of a medical device.
